(19) United States
(12) Patent Application Publication
(10) Pub. No.: US 2004/0170636 A1
(43) Pub. Date: Sep. 2, 2004

(54) Leishmania vaccines

(76) Inventor: Greg Matlashewski, St-Lazare (CA)

Correspondence Address: WOOD, PHILLIPS, KATZ, CLARK & MORTIMER, 500 W. MADISON STREET, SUITE 3800, CHICAGO, IL, 60661, US

(21) Appl. No.: 10/473,446

(22) PCT Filed: Mar. 27, 2002
(86) PCT No.: PCT/CA02/00437

Related U.S. Application Data
(60) Provisional application No. 60/279,423, filed on Mar. 29, 2001.

(51) Int. Cl. A61K 39/00; A61K 39/38

(52) U.S. Cl. 424/184.1

(57) ABSTRACT

The invention provides a DNA vaccine that elicits an immune response in the host in which it is administered, against Leishmania infection. The invention also relates to methods of administering the DNA vaccine. In one embodiment the DNA vaccine contains a vector encoding the A2 gene from Leishmania donovani in a physiologically acceptable medium. The invention further contains a biological adjuvant that includes a vector encoding a selected gene, the selected gene being capable of mediating the degradation of the cellular protein p53.

### BACKGROUND OF THE INVENTION

(a) Field of the Invention

The present invention relates to a vaccine against Leishmania infection, and more particularly to a DNA vaccine that consists of a vector that encodes the A2 virulence gene from Leishmania donovani.

(b) Description of Prior Art

Leishmaniasis is an infectious disease caused by the protozoan parasite Leishmania which affects over 12 million people in 88 countries. There are several principle species of Leishmania that cause different forms of the disease, ranging from self-limiting Cutaneous Leishmaniasis (CL) to Visceral Leishmaniasis (VL), also known as Kala-azar, which is a fatal infection if not treated successfully.

Leishmania is transmitted through the bite of an infected sandfly (Phlebotomus spp.) and it is estimated that over 350 million people are at risk of this infection with an annual incidence of about 2 million new cases (1.5 million cutaneous leishmaniasis, and 0.5 million visceral leishmaniasis). Reservoirs for Leishmania include canine, wild rodents, and human. Within the sandfly host, Leishmania is present as the promastigote and upon entering the mammalian host, it differentiates into the amastigote form where it multiplies exclusively within the phagolysosome compartment of macrophages. Depending on the species of Leishmania, this infection results in a variety of pathologies, ranging from simple skin lesions (cutaneous leishmaniasis), to tissue destruction of the nose and mouth (mucocutaneous leishmaniasis), to fatal visceral disease (visceral leishmaniasis).

Leishmaniasis is difficult to treat and there is increasing resistance developing against the currently available drugs. New disease foci are identified every year in different parts of the world and this may be due to the emerging resistance of sandflies towards insecticides and resistance of the parasite to the existing chemotherapy. In developing and underdeveloped parts of the world, acquired immunosuppressive syndromes (including AIDS) add to the higher risk of leishmaniasis.

Several vaccine clinical trails against cutaneous leishmaniasis have been undertaken however, no such trials have been conducted against visceral leishmaniasis. Most experimental vaccines against leishmaniasis have been either live strains, defined subunit vaccines or crude fractions of the parasite. DNA-vaccination is among the more novel advances in vaccine development and holds promise for use in developing countries because it is relatively simple and inexpensive.

Based on these and other observations, there is clearly an urgent need for vaccine development against this disease and in particular against fatal Kala-azar, and in particular the use of DNA vaccines against the disease.

In U.S. Pat. No. 5,733,778 issued Mar. 31, 1998 to Matlashewski et al:, U.S. Pat. No. 6,133,017 issued Oct. 17, 2000 to Matlashewski et al., U.S. Pat. No. 5,780,591 issued Jul. 14, 1998 to Matlashewski et al. and in U.S. Pat. No. 5,827,671 issued Oct. 27, 1998 to Matlashewski et al., there are described and claimed differentially expressed Leishmania genes and proteins and antibodies raised against proteins, in particular the A2 gene from Leishmania donovani which was thought to have utility as a vaccine. The entire contents of U.S. Pat. No. 5,733,778, U.S. Pat. No. 6,133,017, U.S. Pat. No. 5,780,591 and U.S. Pat. No. 5,827,671, including references, are incorporated herein by reference.

### SUMMARY OF THE INVENTION

The invention relates to specific DNA vaccines that elicit immune responses in the host in which they are administered, against Leishmania infection The invention also relates to methods of administering the DNA vaccines.

In particular the invention relates to a DNA vaccine comprising a plasmid vector encoding the A2 gene from Leishmania donovani in a pharmaceutically acceptable carrier. The invention further comprises a biological adjuvant that includes a plasmid vector encoding a selected gene, the selected gene being capable of mediating the degradation of the cellular protein p53.

The invention also relates to a method of eliciting an immune response against Leishmania infection in a mammal involving administering to the mammal a vaccine that contains a DNA molecule that contains at least one vector that encodes a gene, for example the A2 gene from Leishmania donovani, whereby expression of the gene in one or more cells of the mammal elicits at least one of a humoral immune response or a cell-mediated immune response against Leishmania donovani.

The present invention further provides co-administering a second vector that encodes a selected gene, such as the Human papillomavirus E6 gene, which is capable of mediating the degradation of the cellular protein p53, to inhibit the p53 response in the cells.

The present invention also relates to administering recombinant Leishmania donovani A2 proteins with a suitable adjuvant for immunizing a mammal against Leishmania infection. A2 proteins are composed predominantly of multiple copies of a 10 amino acid repeat sequence.

Finally the present invention relates to use of a DNA vaccine that contains a plasmid vector encoding the A2 gene from Leishmania donovani in a pharmaceutically acceptable carrier for providing immunization against Leishmania donovani.

### BRIEF DESCRIPTION OF THE DRAWINGS

The invention will be better understood with reference to the attached detailed description and to the following Figures, wherein:

FIG. 1 is a graph that shows the infection levels in BALB/c mice following DNA vaccination;

FIG. 2A is graph that shows the relative anti-A2 antibody levels in mice following DNA vaccination;

FIG. 2B shows the western blot analysis of sera for specificity against A2 protein;

FIG. 3A shows the splenocyte proliferation assay for the cellular immune responses in mice receiving DNA immunization with A2 and E6 genes;

FIG. 3B shows the IFN-γ and IL-4 release assay for the cellular immune responses in mice receiving DNA immunization with A2 and E6 genes;

FIG. 3C shows the IgG isotype assay for the cellular immune responses in mice receiving DNA immunization with A2 and E6 genes;

FIG. 4 shows A2 plasmid DNA levels in muscle and spleen derived DNA 2 weeks following DNA immunization;

FIG. 5A shows a Western blot analysis of A2 and p53 protein levels after transfection with the A2 gene alone or in combination with the p53 and E6 genes;

FIG. 5B is a Western blot analysis of A2 protein levels in HT1080 cells transfected with the A2 gene and co-transfected with the A2 and E6 genes;

FIG. 6A is a Western blot analysis of p53 levels in the p53-containing and p53-dvoid HT1080 cells;

FIG. 6B shows a percentage of p53 containing and p53 devoid cells;

FIG. 7 shows Infection levels following A2 protein vaccination as determined by Leishman Donovan Units (LDU);

FIGS. 8A and 8B show the relative anti-A2 antibody levels in mice following A2 protein vaccination;

FIG. 9 shows the proliferation response of spenocytes from mice receiving A2 protein immunization;

FIG. 10A shows an IFN-γ and IL-4 release assay in splenocytes from A2 protein immunized mice;

FIG. 10B is an IgG isotype assay;

FIG. 11 shows infection levels in mice challenged with L. donovani following adoptive transfer of splenocytes from A2 vaccinated mice; and

FIG. 12 shows internalization of amastigotes in the presence of anti-A2 sera.

### DETAILED DESCRIPTION OF THE INVENTION

The present invention will now be described with reference to the above-mentioned Figures.

The present invention relates to the use of a DNA vaccine that contains a vector encoding the A2 gene from Leishmania donovani in a physiologically acceptable medium for providing immunization against any Leishmania species. Any vector that will encode the A2 gene may be used, preferably a vector that contains a cytomegalovirus promoter. In particular the pCDNA3 vector is a suitable vector to be used.

The present invention also relates to a novel approach to increase the effectiveness of DNA-vaccination with the A2 gene against any Leishmania species by co-administering a second vector that encodes a gene that is capable of mediating the degradation of the cellular protein p53, in particular a vector that encodes the Human papillomavirus (HPV) E6 gene. p53 is a cellular protein which is widely accepted as the “guardian of genome”. In response to DNA damage, it is known that p53 levels and activity rise within the cell. Moreover, introduction of plasmid DNA into the nucleus of cells represents a DNA damage signal which effectively induces a strong p53 activation response. The p53 activation response can lead to a variety of cellular effects including apoptosis, cellular senescence, cell cycle arrest, inhibiting the transcription of a variety of promoters including viral promoters, and potentially stimulating. DNA repair mechanisms. Activated p53 could therefore impair DNA-vaccination by several of the above-described mechanisms.

Human papillomavirus (HPV) type 18 E6 protein can effectively. mediate the degradation of p53 through the ubiquitin proteolysis pathway in order to inhibit apoptosis during viral DNA replication in the nucleus of infected cells. It has been demonstrated in transgenic mouse models that expression of E6 could mediate p53 protein degradation in vivo that is indistinguishable from p53 deficiency. The present invention therefore relates to co-administering with the DNA vaccine a vector encoding HPV E6 that will target p53 and thereby increase the effectiveness of the DNA-vaccination.

The present invention further relates to the use of a vector encoding a selected gene that is capable of mediating the degradation of the cellular protein p53, for increasing antibody production in a host. In particular, the use of the Human papillomavirus E6 as the selected gene. The present invention further relates to a method of producing antibodies to a protein in a host comprising the steps of administering to the host a vector encoding a selected gene, the selected gene being capable of mediating the degradation of the cellular protein p53. Any vector can be used that can encode the selected gene of interest, preferably any vector that contains a cytomegalovirus promoter, such as the pCDNA3 vector. Any selected gene that is capable of mediating the degradation of the cellular protein p53 may be used. As an alternative to a selected. gene, any modulator capable of mediating the degradation of the cellular protein p53, such as any cellular MDM protein, may be used.

The present invention also relates to the use of recombinant A2 protein from Leishmania donovani for immunizing a mammal against Leishmania infection. In particular the invention relates to administering recombinant A2 protein with a suitable adjuvant followed by at least-one booster of recombinant A2 protein at a later time.

The following Examples describe vaccination trials using direct DNA-vaccination with the A2 virulence gene and additionally inhibiting the cellular p53 response with human papillomavirus E6. DNA vaccination trials were conducted on female BALB/c mice from 4-6 weeks old, obtained from Charles River Canada.

The current invention is illustrated by the following examples, which are not to be construed as limiting in any way.

### EXAMPLE 1

Leishmania Strain and Source of the A2 Gene

Leishmania donovani donovani Sudanese 1S2D promastigotes were cultured at 26° C. in M199 media (Life Technologies Inc.) supplemented with 10% defined fetal bovine serum (HyClone Laboratories Inc., Logan, Utah.), 25 mM HEPES (pH 6.8), 20 mM glutamine, 10 mg(L folic acid; and 0.1 mM adenosine. Female BALB/c mice (4 to 6 weeks old) were obtained from Charles River Canada.

The A2 gene was originally cloned from L.donovani Ethiopian LV9 strain. and described in detail in, for example in Charest et al., Mol Cell Biol 1994;14:2975-84.

DNA Immunization and Challenge Infection

The pCDNA.3 vector (Invitrogene) was used for the DNA vaccination studies. This vector contains the strong cytomegalovirus (CMV) promoter (Invitrogene) to mediate expression of the A2 and BPV E6 genes. The pCDNA3/A2 expressed the A2 gene, and pCDNA3/E6 encoded the E6 gene and both plasmids were constructed using standard molecular biology procedures. Endotoxin free plasmid DNA was isolated using a Qiagen plasmid purification column (Qiagen Inc, Canada) and dissolved in PBS (pH 7.4). Mice were injected i.m. at two sites in each rear leg thigh skeletal muscle. For the vaccination studies, and the antibody response experiments, each mouse received 100 μg pCDNA/A2+100 μg control pCDNA or 100 μg pCDNA/A2+100 μg control pCDNA/E6 three times at three week intervals. Control mice received only PBS. Mice were bled three weeks following the final injections and serum from the mice in each group (n=4) were pooled. For the vaccination experiment, mice were immunized as above and then challenged three weeks after the final boost and sacrificed for liver biopsies to quantitate levels of infection four weeks after challenge. For challenge infection, 2×10⁸ stationary phase cultured promastigotes of Leishmania donovani 1S2D were injected i.v through tail vein in 100 1 PBS per mice.

For the cell proliferation and cytokine production assays, mice were immunized with 200 g of DNA in 200 1 PBS twice at two weeks intervals. All the mice received the same amount of total DNA, only the quantity of the particular constructs varied. Control mice received 200 g of control vector pCDNA3 and other groups received the following: 100 μg of pCDNA3+100 g of pCDNA3/A2 (A2 expression); 100 μg of pCDNA3+100 μg of pCDNA3/E6 (E6 expression); 100 μg of pCDNA3/A2+100 μg of pCDNA3/E6 (A2 and E6 expression). Two weeks after the second immunization, mice were sacrificed and spleens were isolated. Spleens or serum from mice in the same group (4 per group) were pooled together.

Vaccination Analysis

After four weeks of challenge infection, mice were sacrificed and liver touch biopsies were microscopically examined after fixing and staining the slides with Giemsa , for example as described in Gu et al., Oncogene 1994:9:629-33. LDU. were calculated , for examples as described in Rees et al., Biotechniques 1996;20:102-10, as LDU=(number amastigotes/number liver nuclei)×weight of liver in milligrams. Protection studies were performed in four mice per group and the experiment was repeated twice with similar results.

ELISA

The method-for end point titration is described in Strauss M W, Current Protocols in Molecular Biology, John Wiley & Sons Inc.m, 1998:2.2.1-3. For cytokine capture ELISA of IL-4 and IFN-γ, 5×10⁶/single spleen cell suspensions in RPMI-1640 were stimulated with 10 ng/ml recombinant A2 antigen and culture supernatant were collected after 96 hours. The concentration of IFN-γ and IL-4 in the resulting supernatant was determined, for example as described in Banks L. et al., Eur J Biochem 1986;159:529-34, using biotinylated capture antibody followed by steptavidin conjugated to HRPO (Pharmingen).

Isotype specific antibodies were purchased from Sigma and antigen mediated ELISA were performed according to suppliers instructions. In brief, 0.1 μg of recombinant A2 protein in 100 μl were coated over night at 40° C. in 0.1 M phosphate buffer pH 9.0 and blocked with 200 μl of 3% BSA in PBS-T for 1 hour at room temperature and washed three times with PBS-T. Mouse sera (100 μl) diluted to 1:100 in PBS-T was added to the wells (except for experimental blanks where instead incubated with 3% BSA in PBS-T) and incubated at room temp for two hours then washed three times with PBS-T. Goat-anti mouse isotype antibodies were incubated at 1:1000 dilution for one hour, wash again and incubated with rabbit anti-goat-HRPO conjugate at 1:5000 dilution for 0.5 hours and color was developed with TMB-ELISA. All samples were run in triplicates.

Cell-Proliferation Assay

Single cell suspensions of isolated splenocytes (4×10⁶ cells/ml) were stimulated with 10 ng /ml of recombinant A2 in 200 μl in a 96 well plate at 370° C., 5 % CO₂ for 72 hours and pulsed for additional 18 hours with 1 μCi of [3H] thymidine per well. The plate was harvested and the amount of incorporated [3H] thymidine was measured in a β-counter.

Development of Stable p53-Devoid Cell Lines Expressing HPV-18 E6

Wildtype p53 containing human fibrosarcoma HT1080 cells used in this study were obtained from the American Type Culture Collection (Rockville, Md.) and maintained in Dulbecco's modified Eagles medium (DMEM) containing 10% fetal calf serum and antibiotics. The E6 gene from HPV-18 was removed from the pJ4 vector, for example as described in Gu Z. et al., Oncogene 1994; 9:629-633, and inserted in the pIRESneo vector (Clontech, Mississauga, Ont.) using standard molecular biology procedures. The pIRESneo bicistronic vector has been previously described in Rees S. et al., BioTechn 1996;20:102-110, and contains the CMV promoter followed by a multi-cloning site, the internal ribosome entry site (IRES), the NeoR gene and a polyadenylation site. The resulting plasmid, pIRESneo-E6 was transfected in human epithelial HT1080 cells and selected for stable expression of E6 using G418. Since both E6 and the NeoR genes are expressed on the same bicistronic transcript, G418 selection results constitutive E6 expression. Cells were transfected with 5 μg of pIRESneo or pIRESneo-E6 and selected in G418 as previously described in, for example, Gu Z. et al., Oncogene 1994; 9:629-633.

HT1080 cells and p53 null human Saos-2 cells were also transiently transfected as described above with A2, p53, and E6 expressing plasmids used in the DNA vaccination studies and at various times following transfection, cells were harvested and subjected to Western blot analysis for expression of A2 and p53.

FACS and Microscopic Analysis to Detect GFP

Control p53-containing and p53-devoid HT1080 cells were transfected with the GFP expressing pLantern plasmid as described above and then continuously cultured in D-MEM containing 10% fetal calf serum. At various time intervals, cells were floated in PBS, washed in PBS and resuspended in 0.5 ml PBS and subjected to flow cytometry analysis. Flow cytometry analysis was performed on a FACScan (Becton Dickinson, San Jose, Calif.). An argon ion laser at a wavelength of 488 nm was used to excite GFP with a 518 nm emission filter. The background fluorescence was established using non-transfected control cells.

Nucleic Acid Preparation and Analysis and Western Blot Analysis of p53, and A2

Genomic DNA from muscle and spleen was isolated, for example as described in Strauss, M. W. Current Protocols in Molecular Biology. John Wiley & Sons Inc.1998; 2.2.1-3. PCR was performed on the DNA using 0.75 μg of muscle or spleen DNA template using A2 specific primers (forward:CCACAATGAAGATCCGCAGCG and reverse: CCGGAAAGCGGACGCCGAG). The PCR products were resolved on a 1.2% agarose gel and transferred onto. nylon membranes (Hybond-N, Amersham) and subjected to a Southern blot detection with a A2 specific probe as previously described in Charest, H. et al., Mol. Cell. Biol 1994; 14: 2975-2984.

Western Blot Analysis was carried out as follows: Cells were harvested and placed in lysis buffer (150 mM NaCl, 1.0% NP40, 20 mM Tris pH 8.0) on ice for 30 min and then equal amounts of lysate were incubated in SDS-PAGE sample buffer and subjected to electrophoresis. The resolved proteins were then transferred to a nitrocellulose filter in the presence of 20% V/V methanol, 25 mM Tris, pH 8.2, 190 mM glycine at 30 volts for 12 hours. Filters were washed then incubated directly in anti-p53 Pab1801 hybridoma supernatant or anti-A2 C9 hybridoma supernatant with 5% milk in PBS-T for 2 hours at 22° C. then washed and incubated in the presence of horse radish peroxidase labelled anti-mouse IgG in PBS-T at room temperature for 1 hour. The membrane was then incubated in Amersham ECL detection solution for 1 minute and then exposed to X-ray film followed by autoradiography.

The anti-p53 monoclonal antibody PAb1801 was as previously described in, for example, Banks, L. et al., Eur. J. Biochem 1986;159:529-534. The anti-AS monoclonal antibody was as previously described in, for example, Zhang, W. et al., Mol. Biochem. Parasit 1996;78:79-90.

Statistical Analysis

Significance of difference was examined by student's t-test using “Sigma plot” software arid a value of p<0.05 was considered statistically significant.

DNA-Vaccination with the A2 Gene and Enhanced Protection by Co-Immunization with the E6 Gene

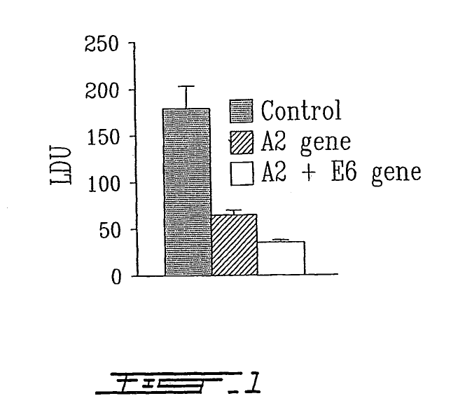

Determination of whether the DNA-vaccination with the A2 gene was protective against infection from L. donovani in BALB/c mice and whether co-immunization with the T:PV E6 gene could alter the protection levels achieved with the A2 DNA-vaccine was undertaken. The-HPV E6 was used to mediate p53 degradation through the ubiquitin proteolytic pathway, as previously described in Thomas, M. et al., Oncogene 1999; 18:7690-7700, in order to suppress the p53 response in cells taking up the DNA vaccine. Mice were immunized with plasmid DNA three times at three week intervals as described in the methods section. Three weeks after the final injection, BALB/c mice were challenged with 2×10⁸ stationary phase L. donovani promastigotes. The degree of protection against infection was evaluated after sacrificing the mice four weeks following the challenge infection. Liver touch biopsies were analyzed for each groups of mice and the mean number of amastigote per liver was determined and the results are presented as Leshman donovan units (LDU). LDU=(number amastigotes/number liver nuclei)×weight of liver in milligrams. FIG. 1 shows the infection levels following DNA vaccination after BALB/c mice were immunized with plasmids encoding A2, A2 plus E6 or PBS three times at 3 week intervals. Three weeks following the final injection, the mice were challenged i.v. with 2×10⁸ Leishmania donovani promastigotes. Four weeks after the challenge infection, mice were killed and Leishman Donovan Units (LDU) was calculated from liver biopsies. The mean LDU ∀ SE is shown in FIG. 1, n=4 mice per group. As shown in FIG. 1, the A2 plasmid immunized mice had reduced the LDU by 65% over the control mice (p=0.0029). Mice co-immunized with the A2 and E6 expression plasmids had 80% reduced LDU over the control group (pp=0.00079). These data demonstrate that DNA-vaccination with the A2 gene provided a significant level of protection against infection. Moreover, co-immunization with the E6 gene to suppress the p53 response provided a greater level of protection than immunization with the A2 gene alone.

Antibody Response Generated Against A2 in the Mice Immunized by DNA-Vaccination

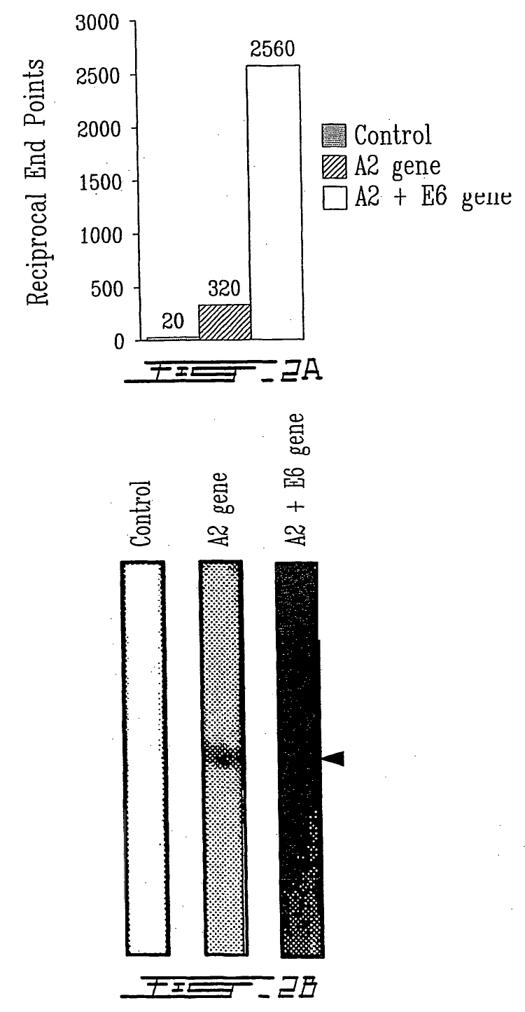

The above observations demonstrated that the A2 gene based DNA-vaccine provided a significant level of protection against infection. The immune response generated against the A2 antigen was characterized as follows. As described in the methods section, mice were immunized three times at three weeks interval, and serum was collected three weeks after the final injection. To determine the titer of anti-A2 antibodies in each immunized group of mice, an ELISA titer 96-well plate was coated with recombinant A2 protein and end point titrations for each group were performed in triplicate starting at 1:20. FIG. 2A shows the anti-A2 antibody. levels determined by reciprocal end point titer. BALB/c mice were immunized as described for FIG. 1 and sera were collected 3 weeks following the final injection, resulting in the representative of two independent experiments and triplicates used for. each sample. As shown in FIG. 2A, the antibody response against A2 was greatest in the mice immunized with a combination of the A2 and the E6 genes (end point=2560), as compared to mice immunized with the A2 gene and a control vector (end point=320). The control group receiving no DNA vaccine showed no anti-A2 response (end point=20).

To confirm that the antibody response was generated against A2, the sera were also tested by Western blot analysis against a recombinant A2 protein. A single well SDS-PAGE gel with recombinant A2 was transferred onto nitrocellulose and stripes were used in immuno-blotting using mice sera at 1:250 dilution. As shown in FIG. 2B, the mice immunized with the A2 gene did generate anti-A2 specific antibodies. Moreover, at this dilution, the sera from the mice co-immunized with both the A2 and E6 genes showed a stronger antibody reaction than other groups. The Western blot data confirmed the ELISA results in demonstrating that the A2 gene DNA-vaccination did generate an anti-A2 antibody response and that this response was significantly increased by co-vaccinating with the E6 gene.

Cellular Th Response Generated Against A2 in the Mice Immunized by DNA-Vaccination

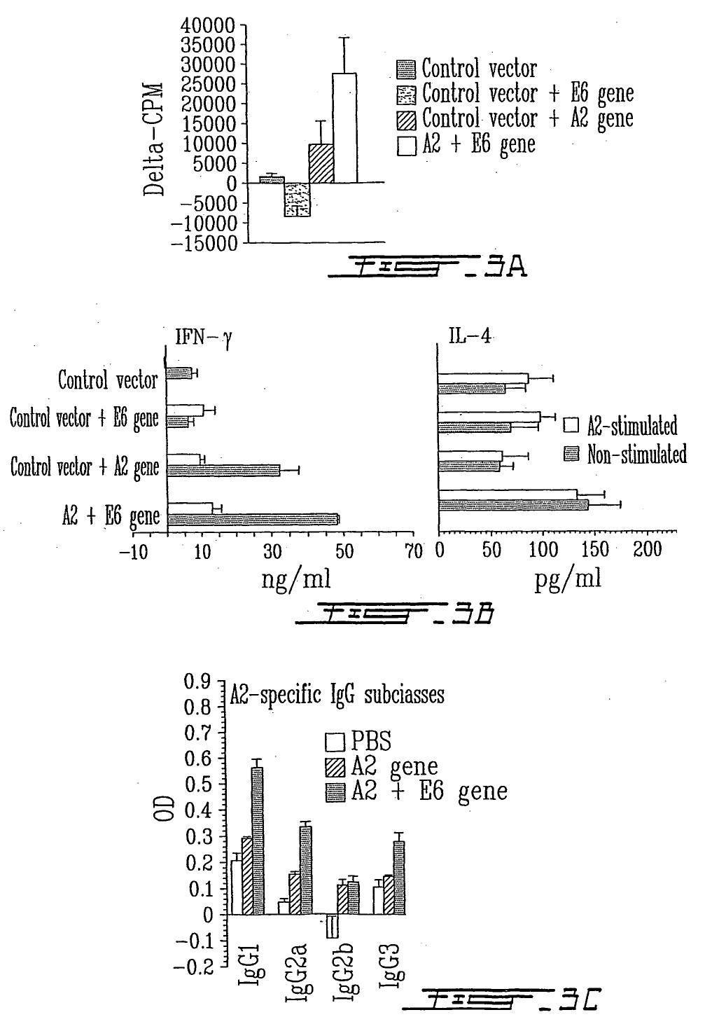

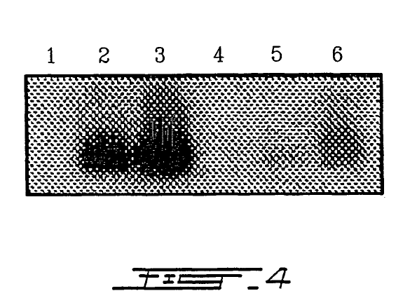

The lymphocyte proliferation response to the A2 antigen in a mixed splenocyte reaction was examined as follows. Mice were immunized twice at two week intervals and spleens were harvested two weeks following the last injection. Lymphocytes from a mixed splenocyte preparation were stimulated with recombinant A2 protein in vitro and thymidine incorporation measured as described in the methods section. FIG. 3A-C shows the cellular immune responses in mice receiving DNA immunization with A2 and E6 genes. FIG. 3A shows a splenoycte proliferation assay. Mice were immunized with the indicated DNAs two times over 2 weeks and then spleens were. collected as described in the methods section above. Splenocytes were stimulated with recombinant A2 protein and thymidine incorporation was determined. Delta CPM represents the difference in counts compared with the corresponding non-stimulated cells. FIG. 3B shows an IFN-γ and IL-4 release assay. Mice were immunized with the indicated DNAs as described in the methods section, splenocytes were stimulated with recombinant A2 protein, and concentrations of released IFN-γ and IL-4 in the culture supernatants were determined. The data is represented as the mean ∀SE. Each sample was examined in triplicate and these results are representative of two experiments. The IFN-γ and IL-4 are represented on different scales. FIG. 3C shows the IgG isotype assay. The A2-specific IgG isotype titre was determined in the serum samples used for the analysis shown in FIGS. 2A and B. The relative subclass titre is represented as OD values and the data is representative of two experiments. As shown in FIG. 3A, thymidine uptake was highest in splenocytes collected from mice co-vaccinated with the A2 gene and the E6 gene. Immunization with the A2 gene alone did however result in splenocyte proliferation in response to stimulation with A2 protein. Thymidine incorporation was negligible over background in the former groups when stimulated with an irrelevant recombinant GST antigen (data not shown). A2, a polymer of 10 amino acid sequences, may bind non-specifically to splenocyte surface from mice which was never exposed to A2 and thus may provide negative signals towards cell survival in vitro. However, it was more prominent in E6 immunized splenocytes.

It has been demonstrated that production of IFN-γ rather than IL-4 determines the degree of resistance of L. donovani infection, as described in Lehmann J. et al., J Interferon Cytokine Res 2000;20(1):63-77. Therefore determination as to whether DNA immunization with the A2 gene resulted in IFN-γ production against the A2 protein was undertaken. As demonstrated in FIG. 3B, Splenocytes from mice vaccinated with the A2 gene secreted significantly higher level of IFN- when stimulated with recombinant A2 protein than splenocytes collected from vector immunized mice (p=0.0054). Moreover, splenocytes from mice co-vaccinated with the A2 and E6 genes secreted higher level of IFN-γ than splenocytes collected from mice vaccinated with the A2 gene alone (p=0.022). In comparison, as shown in FIG. 3B, the release of IL-4 was not significantly higher in the A2 gene immunized mice than control mice following stimulation with recombinant A2 protein. In considering the IFN-γ and IL-4 release observations, these data are consistent with the A2 DNA-vaccination inducing leislmianiacidal response which was further increased when the A2 gene was co-immunized with the E6 gene.

It has been well established that IFN-γ production, a marker of Thl cellular response, directly correlates with a higher IgG2a antibody subclass against the antigen, whereas IL-4, a Th2 marker, is important for generation of IgG1. To further investigate whether the A2 DNA vaccination induced a Th1/Th2 response, the A2 antigen specific IgG subclass antibody levels was examined. For this analysis, mice were immunized three times at three week intervals and the serum collected three weeks after the final injection. The titres of the A2 specific IgG subclasses were then determine as described in the methods section. As shown in FIG. 3C, A2 antigen specific IgG1, IgG2a and IgG3 titres were highest in mice immunized with a combination. of A2 and E6 genes as compared to mice immunized with the A2 gene alone or the control group.

Taken together, the DNA-immunization data show that the A2 gene alone is protective against infection, however co-immunization of the A2 gene together with the E6 gene resulted in a higher level of protection against infection with L. donovani. Likewise, the A2 gene alone was able to stimulate both an antibody response as well as cellular response against recombinant A2 protein, however these immune responses were greater when the A2 gene was co-immunized with the E6 gene. These data show that the A2 gene DNA vaccine can deliver a protective response against L. donovani infection. Moreover, co-vaccination with the E6 gene resulted in the enhanced immunological. response against the A2 gene product. Based on these data, the A2 plasmid maintenance in the injected mice and on heterologous gene expression in cultured cells where p53 levels can be manipulated and quantitated in cells co-expressing E6 was further examined.

A2 DNA Levels in Mice Immunized with Plasmids Encoding A2 and E6

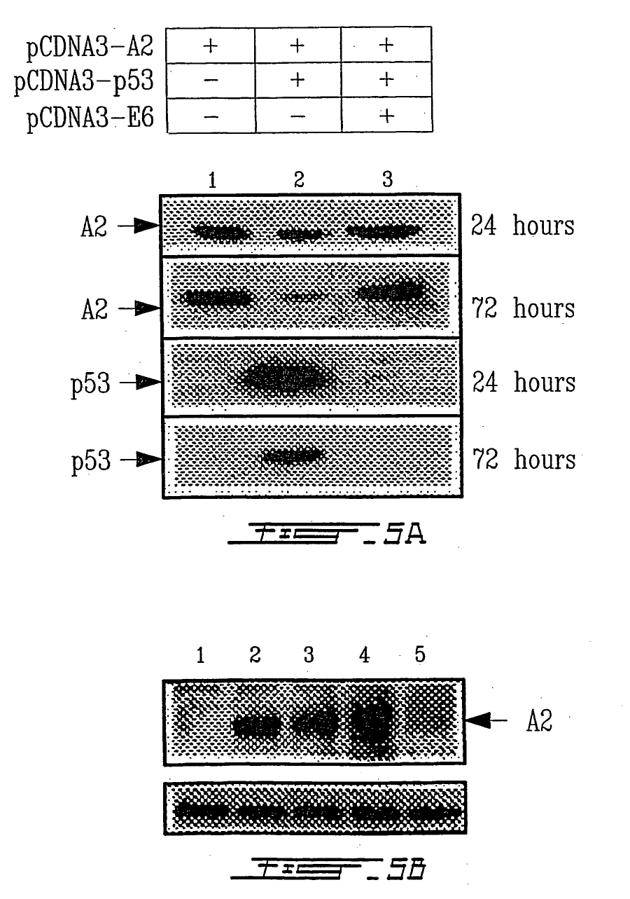

It was determined whether A2-DNA vaccinated mice contained detectable A2 plasmid DNA in the muscle and spleen and what effect E6 would have on the levels of the A2 DNA in these tissues. Mice were immunized twice at two week intervals and total DNA from muscle and spleen was isolated two weeks following the last injection. An equal amount of total DNA from muscle and spleen was used as a template for PCR to amplify A2 sequences using A2 gene specific primers. The limited sensitivity of PCR using this approach led us to visualize and quantitate the amount of A2 specific PCR product by Southern hybridization using an A2 sequence specific probe as described in the methods section. FIG. 4 shows A2 plasmid DNA levels in muscle and spleen derived DNA 2 weeks following DNA immunization A2 genes were amplified by PCR starting with equal amounts of genomic DNA and then the amplified products were subject to Southern blot analysis to semi-quantitate and confirm the presence of the A2 DNA from the samples. Lanes 1-3 in FIG. 4 contain DNA from muscle, lanes 4-6 contain DNA from spleen. Lanes 1 and 4 contain DNA from mice immunized with a control pCDNA3 vector. Lanes 2 and 5 contain DNA from mice immunized with pCDNA3-A2 plus the control pCDNA3 vector. Lanes 3 and 6 contain DNA from mice immunized with pCDNA3-A2 and pCDNA3-E6 vectors. All mice were injected with the same amount of plasmid DNA as described in the previous section. As shown in FIG. 4, mice immunized with a combination of A2 and E6 encoding plasmids contained more A2 gene sequences than immunization with the A2 gene alone and this was more apparent in the spleen than in the muscle. These data confirm that cells within the muscle which took up the A2 DNA vaccine were able to migrate to the spleen. This is consistent with the strong immune response generated against A2 in the vaccinated mice and the significant level of protection obtained when challenged with infection. Although this data is only semiquantitative, it does support the argument that co-immunization with the E6 gene was associated with higher A2 gene copy numbers reaching the spleen. This is consistent with the previous data showing that co-immunization with A2 and E6 genes resulted in better protection against infection and a stronger immune response against A2 than immunization with the A2 gene alone.

The Effect of p53 in Cultured Cells Transfected with Plasmids Expressing A2 or GFP

Although the experiments performed in nice, described above, are appropriate for analyzing the A2 vaccine potential against L. donovani and the immune response against the A2 antigen, it is however difficult to directly examine A2 protein expression and suppression of p53 levels by co-transfection of the E6 gene. Therefore, further analysis was carried out in cultured cell lines to directly examine A2 and p53 levels under defined experimental conditions. Initially, it was determined whether co-expression of p53 affected A2 expression in transfected cells. The A2 expression plasmid used in the vaccination studies above was transfected into p53-negative human Saos-2 cells, both in the presence and absence of a plasmids expressing the p53 and E6 genes. Western blot analysis for A2 and p53 protein levels were then carried out to determine whether co-expression of p53 resulted in reduced expression of A2 and whether E6 could rescue A2 expression in the presence of p5³.

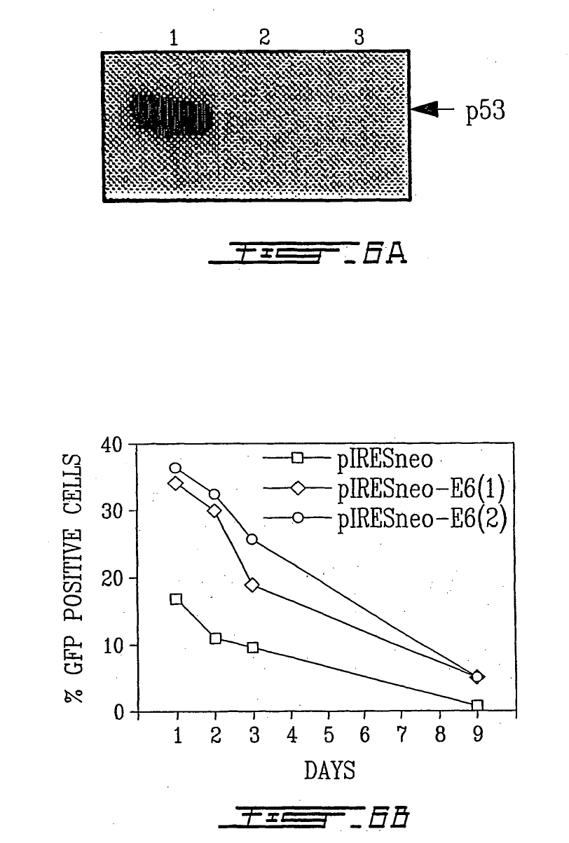

FIGS. 5A and 5B show the effect of p53 on cultured cells expressing A2. FIG. 5A shows the Western blot analysis of A2. and p53 protein levels in 24 hrs and 72 hrs after co-transfection with the A2 gene alone or in combination with the p53 and E6 genes. Cells were transfected with the same amount of plasmid DNA as indicated. Lane 1: pCDNA3-A2 (1 μg), control vector pCDNA3 (2 μg).

Lane 2: pCDNA3-A2 (1 μg), pCDNA3-p53 (1 μg), control vector pCDNA3 (1 μg).

Lane 3: pCDNA3-A2 (1 μg), pCDNA3-p53 (1 μg), pCDNA3-E6 (1 μg). Note that the presence of p53 dramatically reduced the level of A) at 72 hrs post transfection and this was reversed by E6. This is representative of two separate experiments.

FIG. 5B is a Western blot analysis of A2 protein levels in HT1080 cells transfected with the A2 gene and co-transfected with the A2 and E6 gene. The upper blot shows the A2 protein and the lower blot shows an unrelated protein on the blot which serves as an internal control for equal loading. Cells were transfected with the following plasmids. Lane 1, Non-transfected cells. Lane 2, pCDNA3-A2 (5 μg) plus the pCDNA3-E6 vector (5 μg); Lane 3, pCDNA3-A2 (5 μg) plus the control vector pCDNA3 (5 μg); Lane 4, pCDNA3-E6 (5 μg) plus the control vector pCDNA3 (5 μg); Lane 5, Control vector pCDNA3 (10 μg). This is a representative of two separate experiments where the A2 protein level was consistently higher in the cells co-transfected with the E6 gene. As shown in FIG. 5A, the level of A2 protein was similar at 24 and 72 hours following transfection in the cells transfected with the A2 expression plasmid alone (Lane 1) or in combination with both the p53 and E6 expression plasmids (Lane 3).

However, in the cells co-transfected with the A2 and p53 genes in the absence of the E6 gene (Lane 2) there was a noticeable decrease in the level of A2 protein at 24 hours and a further dramatic decrease in A2 protein levels at 72 hours following transfection. As expected, transfection of the p53 expression plasmid resulted in detectable p53 (Lane 2), however cotransfection of the E6 expression plasmid together with the p53 expression plasmid resulted in effective E6-mediated p53 loss (Lane 3). These data highlight two important observations. First, as shown in lane 2, p53 expression effectively reduced A2 levels which was most striking at 72 hours following co-transfection of the A2 and p53 genes. Second, as shown in lane 3, E6 effectively mediated the degradation of p53 and this rescued A2 expression levels to that obtained in the cells transfected with the A2 gene in the absence of the p53 gene. These observations therefore support the argument that suppression of p53 with E6 results in higher levels of plasmid derived A2 following DNA transfection and this is consistent with the DNA vaccination observations reported above.

The reciprocal experiment using HT1080 cells which express an endogenous wildtype p53 was also carried out. Human HT1080 cells were transfected with the A2 and E6 expression plasmids and the level of A2 protein was determine by Western blot analysis 72 hours after transfection. As shown in FIG. 5B, A2 protein was detectable specifically in cells transfected with the A2 expression plasmid (Lanes 2 and 3). There was however a consistently higher level of A2 protein present in the cells co-transfected with the E6 expression plasmid than in cells co-transfected with the control plasmid. This data further argued that suppression of p53 through co-expressing E6, resulted in a higher level of A2 protein expression in those cells taking up the transfected plasmids. Since only about 10 percent of the cells take up the transfected plasmids in this experiment, it was not possible to directly quantitate the suppression of p53 levels in these transfected cells.

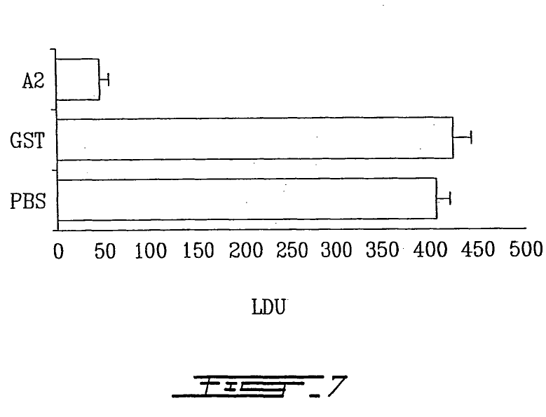

The above experiments were carried out using A2 protein analysis and transient transfections over relatively short time intervals. The study was extended to include an appropriate reporter protein to follow expression levels in live cells over a longer time interval following transfection. For this analysis, HT1080 cells which stably expressed the E6 gene (p53-devoid cells) and control p53-containing cells and transfected with the pLantern plasmid which expresses the green fluorescent protein (GFP) for detection in live cells. The HT1080 p53-devoid cells were developed for this study by transfecting the E6 encoding plasmid vector or the control vector and then placed in G418 to select for cells taking up and expressing the transfected plasmids and several hundred surviving colonies were pooled and used for this analysis. In this manner, pooling colonies obviates clonal variations which typically occurs when analyzing individual clones. Two polyclonal pools of E6 transfected cells were stably selected in this manner and characterised with respect to p53 levels. FIG. 6A is a Western blot analysis of p53-containing and p53-devoid HT1080 cells. Lane 1, wildtype p53-containing cells, Lane 2 and 3. represent two independent p53-devoid cells lines which were selected for E6 expression. FIG. 6B shows the percentage of p53-containing (pIRESneo) and p53-devoid (pIREOneo-E6 [1] and [2]) cells which contained the GFP protein was determined by FACS analysis at the indicated times intervals following transfection with the pLantern plasmid. These are representative data 5 four separate experiments. The E6 expressing cells (pIRESneo-E6 cells lines) contained no detectable p53 protein compared to the control cells which contained abundant levels of p53 (FIG. 6A).

The p53-containing and p53-devoid cells were then transfected with the pLantern plasmid and GFP expression was quantitated over a ten day period in the same population of live cells using FACS analysis. A similar analysis of these cells was performed using fluorescence microscopy (data not shown) and confirmed the FACS results. As shown in FIG. 6B, there was an approximated two fold increase in GFP fluorescence positive cells at the first 24 hour time interval following transfection in the p53-devoid cells compared to the p53-containing cells. Following the first 24 hours, there was also proportionately more GFP positive cells in the p53-devoid cell populations than in the p53-containing cell population. These results are consistent with the transient transfection experiment which likewise showed that co-transfection of E6 was also associated with a higher level of plasmid derived A2 protein.

Taken together, the in vitro experiments support the argument that co-transfection of plasmids encoding E6 in cells containing p53 results in higher levels of heterologous plasmid derived gene products such as A2 or GFP. This is. consistent with the observations that co-vaccination with plasmid DNA expressing E6 and A2 resulted in a superior immune response against A2 and a concomitant better protection against infection against L. donovani. Based on the above data, it appears that the stronger immune response against A2 observed in vivo through co-immunization with an E6 expression vector resulted from higher levels of A2 antigen expression due to suppression of the p53 response.

### EXAMPLE 2

Leishmania Strain and Mice

Leishmania donovani donovani Sudanese 1S2D promastigotes and amastigotes were cultured as described in Zhang W. et al., Proc Natl Acad Sci USA 1997;94:8807-11. Female BALB/c (Lsh^{s}, H-2^{d}) and C57B/6 mice (4 to 6 weeks old) were obtained from Charles River Canada.

A2 Immunization and Challenge Infection

A2 was purified from E.coli BL-21 containing pET16bA2 plasmid. Endotoxin free Recombinant A2 protein was used for vaccination and other studies. Mice were injected i.p. with A2 protein combined with 100ug heat killed Propianibactrium acnes (Elkins.Sinn, Cherry Hill, N.J.) as the adjuvant for the first injection and subsequent boosts were with A2 protein in PBS in the absence of adjuvants. For the vaccination studies, the antibody response experiments, and for passive immunization studies, each mouse received 10 μg of recombinant A2 protein for the first injection and 5 μg each for the 2 boosts with 3 week intervals between each injection. Control mice received only 100 μg heat killed P. acnes as the adjuvant for the first injection and subsequent boosts were with PBS. Mice were bled 3 weeks following the final injections and serum from the mice in each group (n=4) were pooled. For the vaccination experiment, mice were immunized as above and then challenged 3 weeks after the final boost and euthanized for liver biopsies 4 weeks following challenge. For challenge infection, 2×10⁸ stationary phase cultured promastigotes of L. donovaini (1S2D) were injected in the tail vein in 100 μl PBS per mice. For passive immunization, 3 weeks after the final boost 8×10⁸ splenocytes were collected and transferred to naive mice by tail iv. One week after the transfer mice were challenged with 2×10⁸ L. donovani promastigotes and 4 weeks after the challenge infection mice were killed and parasite burden were measured by liver touch biopsy.

For the cell proliferation and cytokine production assays, mice were immunized with 10 μg recombinant A2 protein and 100 μg heat killed P. acnes in the first injection and 5 μg of A2 protein in PBS for 1 boost injection at 2 weeks intervals. Control mice received only 100 μg heat killed P. acnes for the first injection and the subsequent boost was with PBS. Two weeks after the boost, mice were euthanized and spleens were isolated. Spleens from mice in the same group (4 per group) were pooled together.

Vaccination Analysis

Four weeks following challenge infection, mice were euthanized and liver touch biopsies were microscopically examined after fixing and staining the slides with Giemsa, as described in Moore K et al., J. Immunol.1994;152:2930-7. LDU (Leishman Donovan Unit) were calculated as LDU=(number amastigotes/number liver nuclei)×weight of liver in milligrams, as described in Stauber LA. Some physiological aspects and consequences of parasitism. W. H. Cole, ed. Rutgers University Press. New Brunswick, N.J. 1995. p. 76. Protection studies were performed in 4 mice per group and the experiment was repeated 3 times with similar results.

ELISA

The method for end point titration was performed as described in Raj V S et al., Am J Trop Med Hyg. 1999;61:482-7.

For cytokine capture ELISA of W-4 and IFN-γ5×10⁶/single spleen cell suspensions in RPMI-1640 were stimulated with 50ng/ml recombinant A2 antigen and culture supernatant were collected after 96 hours. The concentration of IFN-γ and IL-4 in the resulting supernatant was determined as described in Dotsika E. et al., Scand J Immunol, 1997;45:261-8, using biotinylated capture antibody followed by steptavidin conjugated to HRPO (Pharmingen).

Isotype specific antibodies were purchased from Sigma and antigen mediated ELISA were performed according to suppliers instructions. In brief, 100 ng of recombinant. A2 protein in 100 μl were coated over night at 4° C. in 0.1 M phosphate buffer pH 9.0 and blocked with 200 μl of 3% BSA in. PBST for 1 hour at room temperature and washed 3 times with PBST. Mouse sera (100 μl) diluted to 1:100 in PBST was added to the wells and incubated at room temp for 2 hours then washed 3 times with PBST. Goat-anti mice isotype antibodies were incubated at 1:1000 dilution for 1 hour washed again and rabbit anti-goat-HRPO at 1:5000 dilution was incubated for 0.5 hours and the color was developed with TMB-ELISA. All samples were run in triplicates.

Cell Proliferation Assay

Single cell suspensions of isolated splenocytes (4×10⁶ cells/ml) were stimulated with 0.5 μg/ml of recombinant A2 in 200 μl in a 96 well plate at 37° C., 5% CO₂ for 72 hours and pulsed for additional 18 hours with 1 μCi of [3H] thymidine per well. The plate was harvested and the amount of incorporated [3H] thymidine was measured in a β-counter. Results are represented as the difference in counts obtained between the A2 stimulated and non-stimulated controls.

Western Blot Analysis of A2

The SDS-PAGE (12%) was run with 1 μg of Recombinant A2 protein in each lane. The resolved proteins were then transferred to a nitrocellulose filter in the presence of 20% V/V methanol, 25 mM Tris, pH 8.2, 190 mM glycine at 30 volts for 12 hours. Filters were washed then incubated directly in anti-A2 C9 hybridoma supernatant, for example as described in Zhang W et al., Mol Biochem Parasit 1996;78:79-90, with 5% milk in PBS-T for 2 hours at 22° C. then washed and incubated in the presence of horse radish peroxidase labeled anti-mouse IgG in PBS-T at room temperature for 1 hour. The membrane was then incubated in Amersham ECL detection solution for 1 minute and then exposed to X-ray film followed by autoradiography.

Infection of Macrophages with Amastigotes

Bone marrow derived macrophages (BMM) were obtained from femurs of 6 to 8 weeks old female BALB/c mice as described in Jardim A. et al., J Immunol. 1991;147(10):3538-44. Quiescent BMM (10⁶ cells/ml) were infected with cultured amastigotes at a ratio of 1:1 amastigote per macrophage for 24 hours in polystyrene tubes. The infected BMMs were washed extensively for 4 times with 50 volume PBS at 900 rpm for 10 minutes. Internalization of parasites was measured by microscopic count of Giemsa-stained cytocentrifuged slides. The sera were decomplimented by incubating at 65° C. for 2 hours in a water bath.

Statistical Analysis

Significance of difference was examined by student's t-test using “GraphPad PRISM” (version 3.02) software with 99% confidence intervals and a value of p<0.05 was considered statistically significant.

Immunization with A2 Protein Protects Mice from L. donovani Infection

It was determined whether immunization with the recombinant A2 protein was protective against infection from L. donovani in BALB/c mice. As described in the introduction, the A2 protein is a L. donovani amastigote specific gene product which is highly expressed in infected macrophages. Mice were immunized with recombinant A2 protein as described in the Methods section and 3 weeks after the final injection; BALB/c mice were challenged with L. donovani promastigotes. The degree of protection against infection was evaluated by amastigote levels in the liver touch biopsies and represented as Leshman Donovan units (LDU). FIG. 7 shows infection levels following A2 protein vaccination as determined by Leishman Donovan Units (LDU). BALB/c mice were immunized with recombinant A1 or recombinant GST protein 3 times at 3-week intervals as described in the Methods. Three weeks following the final injection, the mice were challenged i.v with 2×10^{δ} L. donovani promastigotes. Four weeks after the challenge infection, mice were killed and LDU was calculated from liver biopsies. The mean LDU±SE is shown (n=4 mice per group). This result is the representative of 3 independent experiments. As shown in FIG. 7, A2 protein immunization had reduced the LDU by 89% over the control mice or recombinant GST protein immunized mice (p<0.0001). These data demonstrate that vaccination with the recombinant A2 antigen provided a significant level of protection against infection.

High Specific Antibody Titer Generated in Mice Immunized with A2

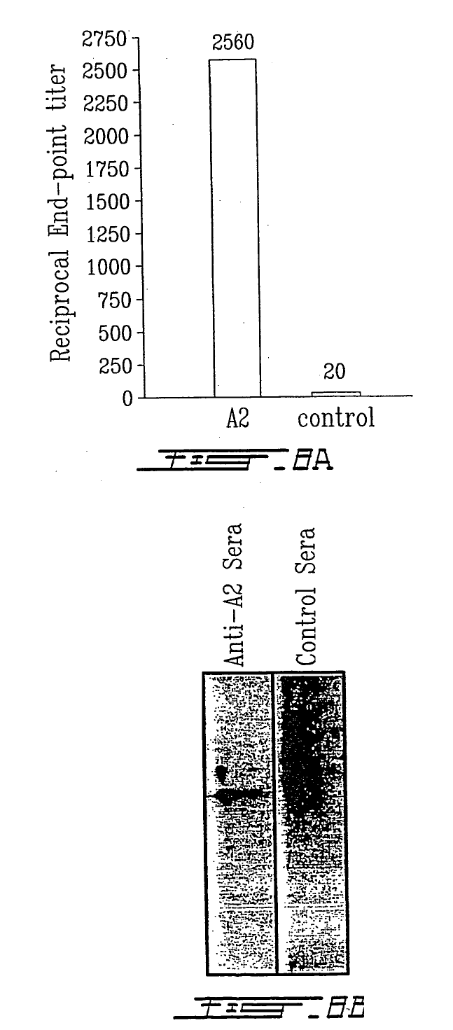

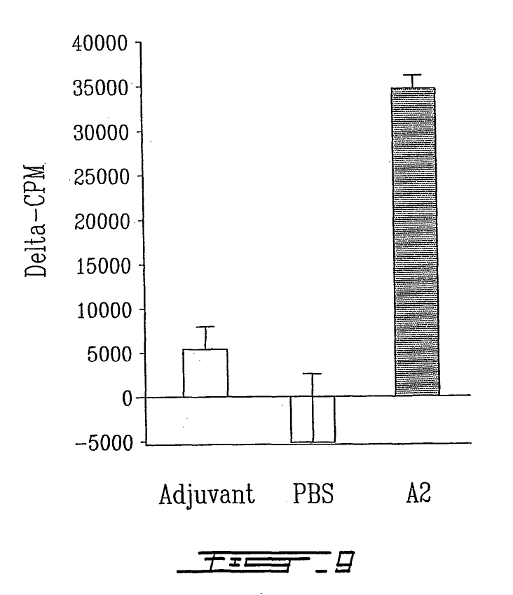

The above observations demonstrated that the recombinant A2 protein immunization provided a significant level of protection against infection. The immune response generated against the A2 antigen was determined. To determine the titer of anti-A2 antibodies in each immunized group of nice, an ELISA end point titration was performed. FIG. 8 shows the relative anti-A2 antibody levels in mice following A2 protein vaccination. FIG. 8A shows anti-A2 antibody levels that were determined by reciprocal end point titer for BALB/c mice that were immunized as described in FIG. 7 and. This result is the representative of 2 independent experiments and triplicates were used for each sample. FIG. 8B is a Western blot analysis of serum for specificity against A2 protein. Serum were used at 1:500 dilution on ng of recombinant A2 protein per lane. As shown in FIG. 8A, the antibody response against A2 was much higher in the mice immunized with At antigen with a reciprocal end point titre reaching 2560 as compared to mice immunized with adjuvant only.

To confirm that the antibody response was generated against A2, the sera (1:500 dilution) were also tested by Western blot analysis against recombinant A2 protein. As shown in the FIG. 8B, the sera from the mice immunized with recombinant A2 protein demonstrated a specific anti-A2 antibody response. These Western blot data confirmed the ELISA results in demonstrating that A2 vaccination did generate a strong anti-A2 antibody response:

Antigen Specific Splenocyte Proliferation in the Mice Immunized with Recombinant A2 Antigen

The lymphocyte proliferation response to A2 antigen in a mixed splenocyte reaction was examined, as described in Methods. Lymphocytes from a mixed splenocyte preparation were stimulated with recombinant A2 protein in vitro and thymidine incorporation measured. FIG. 9 shows the proliferation response of spenocytes from mice receiving A2 protein immunization. Mice were immunized with A2 as described in Methods and spleens were collected following the final immunization. Spenocytes were stimulated with recombinant A2 and thymidine incorporation was measured. Delta CPM represents the difference in counts compared with the corresponding non-stimulated cells. Control mice received either adjuvant or PBS. As shown in FIG. 9, thymidine uptake was much higher in splenocytes collected from mice vaccinated with the recombinant A2 antigen. Immunization with the adjuvant alone or PBS resulted in minimal splenocyte proliferation in response to stimulation with A2 protein Thymidine incorporation was also negligible over background in the former groups when stimulated with an irrelevant recombinant GST antigen (data not shown).

Induction of IFN-γ Production in Response to A2 Protein Stimulation in Splenocytes of Immunized Mice

It has been established that protection against L. donovani infection requires an IFN-γ activated immune response generated against the parasite, as described in Carvalho E M et al., J Immunol. 1994;152:5949-56 and Carvalho E M et al., J Infect Dis. 1992;165:535-40, and production of IFN-γ rather than IL4 determines the degree of resistance of L. donovani infection, as described in Lehmann J et al., J Interferon Cytokine Res. 2000;20:63-77. It was determined whether immunization with the recombinant A2 protein resulted in increased IFN-γ or IL-4 production in response to A2 challenge.

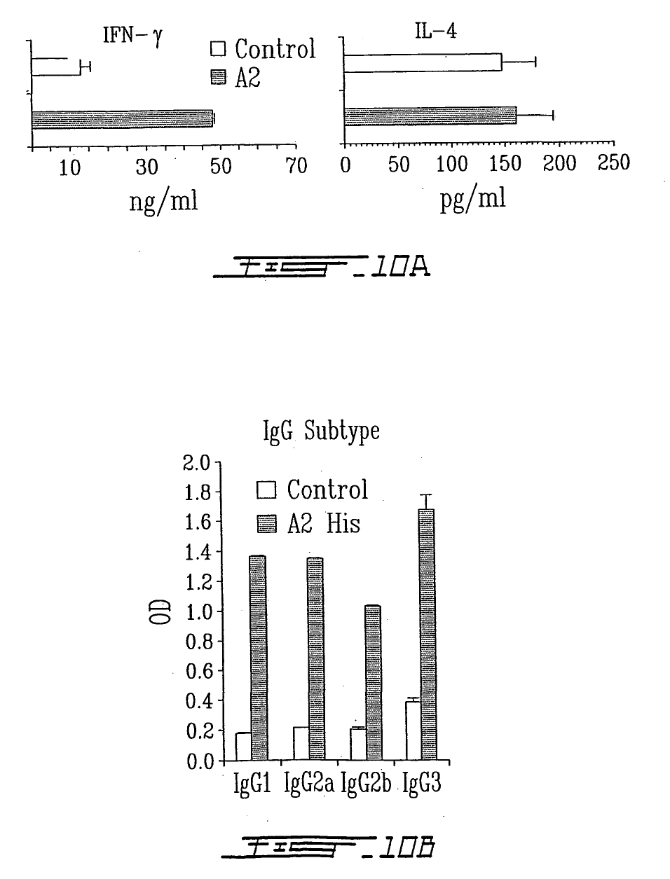

FIG. 10A shows an IFN-γ and IL-4 release assay in splenocytes from A2 protein immunized mice. Mice were immunized with A2 as described in Methods. Splenocytes were stimulated with recombinant A2 for 96 hours and concentrations of IFN-γ and IL-4 in the culture supernatants was determined. The data is represented as the mean±SE. Each sample was examined in triplicate and these results are representative of 2 experiments. Note that the IFN-γ and IL-4 are represented on different scales. FIG. 10B is an IgG isotype assay. The A2 specific IgG isotype titre was determined by ELISA. The relative subclass titre is represented as OD values and the data is representative of 2 experiments. Control mice received only adjuvant as described in Methods. As shown in FIG. 10A, splenocytes from nice vaccinated with A2 secreted significantly higher level of IFN-γ(p<0.0001) when stimulated with A2 than splenocytes collected from control mice. Moreover, the release of IL-4 was not significantly higher in the recombinant A2 antigen immunized mice than control mice following stimulation with A2.

It has been previously shown that IFN-γ production, a marker of Th1 cellular response, directly correlates with a higher IgG2a antibody subclass against the antigen, as described in Snapper C M et al., Science 1987;236:944-7, whereas IL-4, a Th2 marker, is associated with generation of IgGl, as described in Warren H S et al., Annu Rev Immunol. 1986;4:369-88. The A2 antigen specific IgG subclass antibody levels in immunized mice as described in Methods were investigated. As shown in FIG. 10B, all of the A2 antigen specific IgG subclass titres were significantly higher in mice immunized with recombinant A2 protein than in the control group. These data argue that A2 immunization resulted in stimulating both Th1 and Th2 response against the A2 protein.

The A2 antigen immunization data show that the A2 is protective against L. donovani infection and was able to stimulate both an antibody response as well as induce IFN-γ production in response to recombinant A2 protein. These data strongly argue that the A2 antigen has the prerequisite characteristics for delivering a protective immune response against L. donovani infection.

Adaptive transfer of splenocytes from A2 vaccinated mice protects against L. donovani infection.

Protection against L. donovani infection is thought to be predominantly T-cell mediated as demonstrated by adaptive transfer of immune spleen cells to naive mice, as described in Rezai H R et al., Clin Exp Immunol. 1980;40:508-14. Thus adaptive transfer of spleen cells from A2-immunized mice was carried out in both BALB/c and C57BL/6 mice as described in Methods.

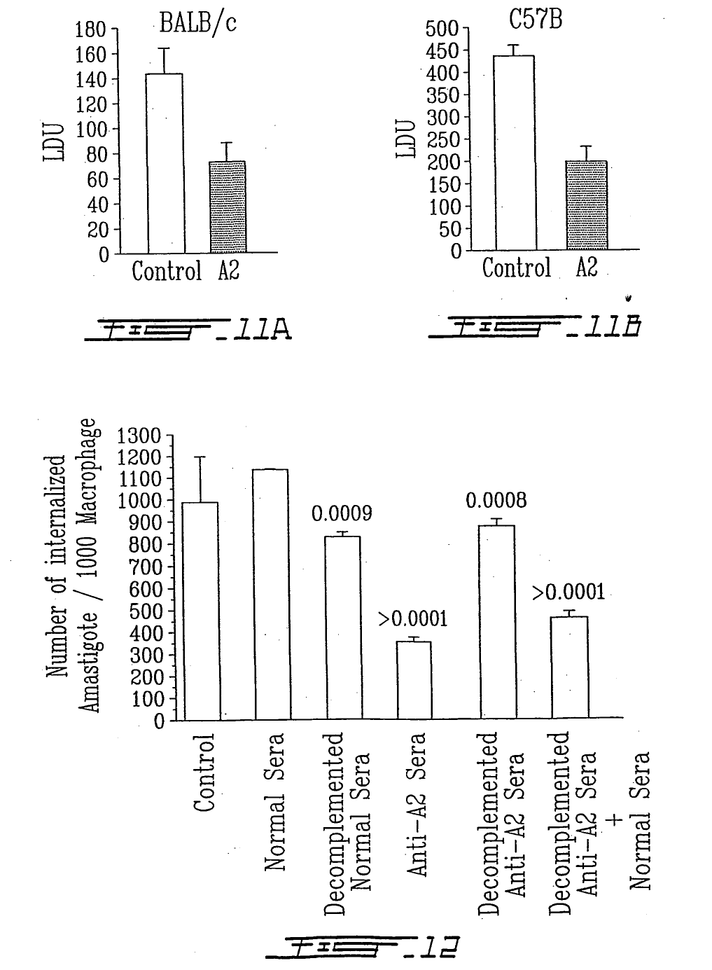

FIG. 11 shows infection levels in mice challenged with L. donovani following adoptive transfer of splenocytes from A2 vaccinated mice. BALB/c and C57B/6 mice were immunized with A2 protein and 3 weeks following the final boost, spleen cells were collected and transferred to naive mice. One week after the transfer, mice were challenged with L. donovani promastigotes and 4 weeks after the challenge infection, mice were killed and Leishman Donovani Units (LDU) was calculated from liver biopsies. The mean LDU±SE is shown (n=4 mice per group). This result is the representative of 2 independent experiments. As shown in the FIG. 11, mice demonstrated a significant level of protection when passively immunized with spleen cells from A2 vaccinated mice in comparison to the control group of mice which received spleen cells from adjuvant immunized mice. The LDU was reduced by 50% (p=0.0215) and 55% (p=0.0044) for BALB/c and C57BL/6 mice respectively. These results confirm that irrespective of the strain of mice, A2 antigen passive immunization imparts significant protection against challenge infection.

Anti-A2 Antibodies and Complements Block Amastigote Internalization by Macrophages In Vitro

Bone marrow derived macrophages (BMMs) from BALA/c mice represents an appropriate cell type to measure-infection by Leishmania in vitro. The in vitro model system was used to measure infection with L. donovani amastigotes in macrophages in the presence of anti-A2 antibodies. This was carried out both in the presence and absence of viable complement. BMMs were incubated with the same number of L. donovani amastigotes in the presence of 1:50 dilution of the various sera combinations.

FIG. 12 shows internalization of amastigotes in the presence of anti-A2 sera. Bone marrow derived macrophages (10⁶ cells/ml) were infected with amastigotes for 24 hours and internalization of parasites were measured. Prior to infection, the amastigotes were incubated with indicated sera samples or control (no-sera). The result is represented as number of internalized amastigotes per 1000 macrophages. The P-values of t-test indicated on each bar are in comparison with values obtained from normal sera treatment. The mean±SE is shown (n=3). This result is the representative of 3 independent experiments. As shown in FIG. 12, there was a significant reduction in L. donovani infection in the presence of anti-A2 sera. However, when the anti-A2 sera was decomplemented, the internalization of amastigotes was significantly increased to levels similar to the control. When decomplemented anti-A2 sera was reconstituted with normal mouse sera as a source of complement the internalization was again significantly reduced. Similar observations were made using anti-A2 monoclonal antibodies where the addition of compliment to these antibodies also reduced the levels of infection (data not shown). These data argue that the A2 antisera in the presence of complement can reduce the viability of amastigotes resulting in a reduction in infection of macrophages.

While the embodiment discussed herein is directed to a particular implementation of the invention, it will be apparent that variations of this embodiment are within the scope of the invention.

What is claimed is:

1. A method of eliciting an immune response against Leishmania donovani infection in a mammal, the method comprising administering a vector comprising an isolated nucleotide sequence encoding at least one A2 gene from Leishmania donovani, and transcriptional and translational regulatory sequences operably linked to the isolated nucleotide sequence, whereby expression of the gene in one or more cells of the mammal elicits at least one of a humoral immune response and a cell-mediated immune response against any Leishmania species.

2. The method of claim 1, wherein the nucleotide sequence further encodes Human papillomavirus E6 gene.

3. A method of eliciting an immune response against Leishmania donovani infection in a mammal, the method comprising administering to the mammal a DNA vaccine, the DNA vaccine comprising at least one vector, the at least one vector encoding at least A2 gene from Leishmania donovani, whereby expression of the gene in one or more cells of the mammal elicits at least one of a humoral immune response and a cell-mediated immune response against any Leishmania species.

4. The method of claim 3, wherein the at least one vector further encodes Human papillomavirus E6 gene.

5. The method of claim 3, wherein the method further comprises co-administering a second vector, the second vector encoding Human papillomavirus E6 gene.

6. The method of claim 3, wherein the at least one vector is a vector that contains a cytomegalovirus promoter.

7. The method of claim 6 wherein the vector is pCDN3 vector.

8. The method of claim 5, wherein the second vector is a vector that contains a cytomegalovirus promoter.

9. The method of claim 8, wherein the vector is pCDNA3 vector.

10. The method of claim 3, wherein the method further comprises administering to the mammal a booster containing recombinant A2 protein and a suitable adjuvant.

11. A method of optimising a DNA vaccine comprising co-administering the DNA vaccine with a vector encoding a selected gene, the selected gene being capable of mediating degradation of cellular protein p53.

12. The method of claim 11, wherein the gene is Human papillomavirus E6 gene.

13. A DNA vaccine against Leishmania infection comprising a plasmid vector encoding A2 gene from Leishmania donovani in a pharmaceutically acceptable carrier.

14. The DNA vaccine of claim 13, wherein the vaccine further comprises a biological adjuvant.

15. The DNA vaccine of claim 14, wherein the biological adjuvant comprises a plasmid vector encoding a selected gene, the selected gene being capable of mediating degradation of cellular protein p53.

16. The DNA vaccine of claim 15, wherein the selected gene is Human papillomavirus E6 gene.

17. A DNA vaccine comprising a plasmid vector encoding A2 gene from Leishmania donovani and a biological adjuvant in pharmaceutically acceptable carrier.

18. The DNA vaccine of claim 17, wherein the biological adjuvant comprises a vector encoding a selected gene, the selected gene being capable of mediating degradation of cellular protein p53.

19. The DNA vaccine of claim 18, wherein the gene is Human papillomavirus E6 gene.

20. A DNA vaccine comprising a plasmid vector encoding A2 gene from Leishmania donovani and the Human papillomavirus E6 gene in a pharmaceutically acceptable carrier.

21. A plasmid vector comprising a DNA sequence encoding A2 gene from Leishmania donovani.

22. A plasmid vector comprising a DNA sequence encoding A2 gene from Leishmania donovani and Human papillomavirus E6 gene.

23. The use of a plasmid vector in a vaccine for eliciting an immune response against Leishmania donovani infection in a mammal, wherein said plasmid comprising a DNA sequence encoding A2 gene from Leishmania donovani.

24. The use of a plasmid vector in a vaccine for eliciting an immune response against Leishmania donovani infection in a mammal, wherein said plasmid comprising a DNA sequence encoding A2 gene from Leishmania donovani and Human papillomavirus E6 gene.

25. The use as claimed in any of claims 23 or 24 wherein the vector is pCDNA3 vector.

26. A method of-eliciting an immune response against Leishmania infection in a mammal, the method comprising administering to the mammal an initial dose of recombinant A2 protein and a pharmaceutically suitable adjuvant.

27. The method of claim 26, wherein the method further comprises administering to the mammal at least one dose of recombinant A2 protein and at least one of a suitable adjuvant or PBS, at a later time from administration of the initial dose.

28. The use of recombinant Leishmania donovani A2 protein in inhibiting and/or preventing Leishmania infection in a mammal.

29. The use of Human papillomavirus E6 gene to mediate p53 degradation for increasing antibody production in a host.

30. The use of a vector for increasing antibody production in a host wherein said vector encoding a selected gene, the selected gene being capable of mediating degradation of cellular protein p53.

31. The use of a vector encoding Human papillomavirus E6 gene for mediating p53 degradation and increasing antibody production in a host.

32. A method of producing antibodies to a protein in a host comprising the steps of administering to the host a vector encoding a selected gene, the selected gene being capable of mediating degradation of cellular protein p53.

33. A method of producing antibodies to a protein in a host comprising the steps of administering to the host a vector encoding a mediator, the mediator being capable of mediating degradation of cellular protein p53.

34. The use of a mediator to mediate p53 degradation in a host for increasing antibody production in the host.


SEQUENCE LISTING (2 sequences)

SEQ ID NO: 1 - DNA, 21 bp, Artificial Sequence
  primer forward
ccacaatgaagatccgcagcg

SEQ ID NO: 2 - DNA, 19 bp, Artificial Sequence
  primer reverse
ccggaaagcggacgccgag